(19) United States
(12) Patent Application Publication
(10) Pub. No.: US 2002/0177862 A1
(43) Pub. Date: Nov. 28, 2002

(54) Second generation coil fastener applier with memory ring

(76) Inventors: Ernest Aranyi, Easton, CT (US); Frank M. Rende III, Westport, CT (US)

Correspondence Address: U.S. Surgical, a division of, TYCO HEALTHCARE GROUP LP, 150 Glover Avenue, Norwalk, CT, 06856, US

(21) Appl. No.: 10/053,889

(22) Filed: Oct. 23, 2001

Related U.S. Application Data
(60) Provisional application No. 60/130,922, filed on Apr. 23, 1999.

(30) Foreign Application Priority Data
Apr. 21, 2000 PCT/US00/10921

(51) Int. Cl. A61B 17/08

(52) U.S. Cl. 606/153

(57) ABSTRACT

There is provided an endovascular fastener applier for fastening a vascular graft to a vessel with at least one fastener. The applicator generally includes a tubular body configured for positioning within the vessel and an expandable portion adjacent the tubular body. A fastener applying head is movably mounted within the expandable portion. The applicator further includes a handle assembly mounted on a proximal end of the tubular body. The handle assembly generally includes controls for expanding the expandable portion, pivoting and rotating the fastener applying head and for driving a fastener out of a fastener driving head. Preferably, the applicator also includes a storage chamber extending, from a distal end of the expandable portion, and containing a plurality of fasteners.

### CROSS REFERENCE TO RELATED APPLICATIONS

This patent application claims the benefit of U.S. Provisional Application Serial No. 60/130,922, filed Apr. 23, 1999, the entire contents of which are incorporated by reference herein.

### BACKGROUND

1. Technical Field

This disclosure relates generally to vascular grafts for intraluminal delivery and, in particular, to a method and apparatus for repairing diseased or damaged sections of a vessel by fastening a prosthesis within the vessel.

2. Description of Related Art

Diseased or damaged blood vessels often cause weakening of the vessel wall resulting in an aneurysm whereby a blood vessel and especially an artery have a section of abnormal blood-filled dilation. For example, an abdominal aortic aneurysm is a sac caused by an abnormal dilation of the wall of the aorta, a major artery of the body as it passes through the abdomen.

The abdominal aortic aneurysm usually arises in the infrarenal portion of the arteriosclerotically diseased aorta, for example, below the kidneys. Left untreated, the aneurysm will eventually cause rupture of the sac with ensuing fatal hemorrhaging in a very short time. High mortality associated with rupturing led the state of the art into trans-abdominal surgical repair of abdominal aortic aneurysms.

Surgery involving the abdominal wall, however, is a major undertaking with associated risks. This type of surgery, in essence, involves replacing the diseased and aneurysmal segment of blood vessel with a prosthetic device which typically is a synthetic tube, or graft, usually fabricated of either DACRON™, TEFLON™, or other suitable material.

The present state of the art for intraluminal repair of a vessel does not fasten a prosthesis to the remaining aortic wall. For example, U.S. Pat. Nos. 5,571,171 and 5,571,173 disclose a method and apparatus for treating an abdominal aortic aneurysm by supplying a prosthesis or an aortic graft for intraluminal delivery that does not fasten the graft to the remaining aortic wall.

Presenting an aortic graft through the aorta by intraluminal delivery avoids major invasive surgery. The '171 and '173 patents disclose an aortic graft that is delivered intraluminally to the aneurysm site. The aortic graft is secured to the remaining aortic wall by a balloon that is inflated thereby causing the graft to contact and adhere to the remaining aortic wall.

The major disadvantages related to the combination of endovascular expanders, such as a balloon or stent, and prosthesis is the dilation of the natural artery with consequent migrations and periprosthetic losses. Upon withdrawal of the expander, the tissue is caused to collapse and the prosthesis disengages from the remaining aortic wall and tends to migrate to a location away from the aneurysm site to be repaired. The migration and movement of the disengaged aortic graft would then obstruct the affected vessel. The migration and movement of the aortic graft requires further treatment on the patient to remove the failed attempt to attach the aortic graft to the remaining aortic wall.

Further treatment may include major surgery that is hazardous and traumatic to the patient. Major surgery to remove the aortic graft defeats the benefits of intraluminal delivery of the aortic graft. The current state of the art does not disclose a fastener applicator that intraluminally delivers a vascular graft and endoluminally applies internal fasteners to fasten a prosthesis in place.

Accordingly, there is a present need for a fastener applicator that intraluminally delivers a vascular graft to a site within a vessel and applies fasteners to pass through both a prosthesis and the thickness of a vessel wall. The fastened prosthesis should also have the capability of following dilation of a vessel.

An exemplary instrument also suitable for use in installing a novel graft assembly is disclosed in provisional patent application No. 60/101,050 entitled, “Endovascular Fastener Applicator”, filed with the U.S. Patent and Trademark Office on Sep. 18, 1998 and in International Application No. PCT/US99/21414, filed Sep. 17, 1999, the entire disclosures of which are incorporated by reference herein.

### SUMMARY

There is provided an endovascular fastener applicator for endoluminally fastening a prosthetic graft to a vessel with at least one fastener. The applicator generally includes a tubular body configured for positioning within a vessel and an expandable portion disposed adjacent a distal end of the tubular body and being expandable to support a prosthetic in contact with an inner surface of a vessel. A fastener applying head is rotatably mounted on the distal end of the tubular body and is movable between a load position longitudinally aligned with the tubular and a firing position oriented approximately 90° with respect to the tubular body. The applicator also includes a handle assembly mounted on a proximal end of the tubular body and having a first control to expand the expandable portion, a second control to pivot the fastener driving head to the firing position and rotate the fastener driving head about the longitudinal axis of the tubular body.

The handle assembly further includes a third control to move a fastener out of the fastener driving head and into tissue and move a fastener carrying slider into engagement with the tissue.

Preferably, the fastener is a helical coil fastener. The applicator has a storage chamber extending from a distal end of the expandable portion, the storage chamber containing at least one helical coil fastener.

Preferably, the third control is connected to the fastener carrying slider by a wire formed of a shape memory material.

Alternately, the fastener driving head is configured to drive a conventional staple into tissue.

### BRIEF DESCRIPTION OF THE DRAWINGS

Various embodiments are described herein with reference to the drawings, wherein:

FIG. 1 is a perspective view of a first embodiment of a coil fastener applier for use in installing a prosthetic graft assembly;

FIG. 2 is a perspective view of the distal end of the coil fastener applier with an onion assembly in an expanded condition;

FIG. 3 is a perspective view of the distal end of the coil fastener assembly with the onion assembly in the expanded condition and a staple driving head pivoted 90°;

FIG. 4 is a view similar to FIG. 3 illustrating a slider extending from the staple driving head and a staple being ejected from the slider;

FIGS. 5-7 are views of the distal end of the coil fastener applier with the onion assembly expanded;

FIGS. 8-10 are views of the distal end of coil fastener applier with the staple driving head pivoted 90°;

FIG. 11 is a side view, partially shown in section, of the fastener driving head pivoted 90°;

FIG. 12 is a perspective view illustrating a driving wire;

FIG. 13 is a perspective view of a slide associated with the staple driving head;

FIG. 14 is a perspective view of the instrument handle assembly;

FIG. 15 is a top plan view of the instrument handle assembly;

FIG. 16 is a perspective view of the first embodiment prior to use;

FIG. 17 is a perspective view of the first embodiment during testing;

FIG. 18 is a perspective view, partially shown in section, illustrating the distal end of the coil fastener applier positioned within an aneurysm site;

FIG. 19 is a perspective view, similar to FIG. 16, with the staple driving head pivoted 90° and a staple being inserted through graft material into a vessel wall;

FIG. 20 is a perspective view of a novel graft assembly positioned about the distal end of a coil fastener applier;

FIGS. 21-22 are perspective views of an alternative staple driving head;

FIGS. 23-24 are perspective views of the staple driving head of FIG. 21 without the outer casing;

FIG. 25 is a perspective view of the distal end of a coil fastener applier with a further alternative fastener applying head;

FIG. 26 is a perspective view of the alternative fastener driving head;

FIGS. 27-28 are further alternative views of the staple driving head.

### DETAILED DESCRIPTION OF THE PREFERRED EMBODIMENTS

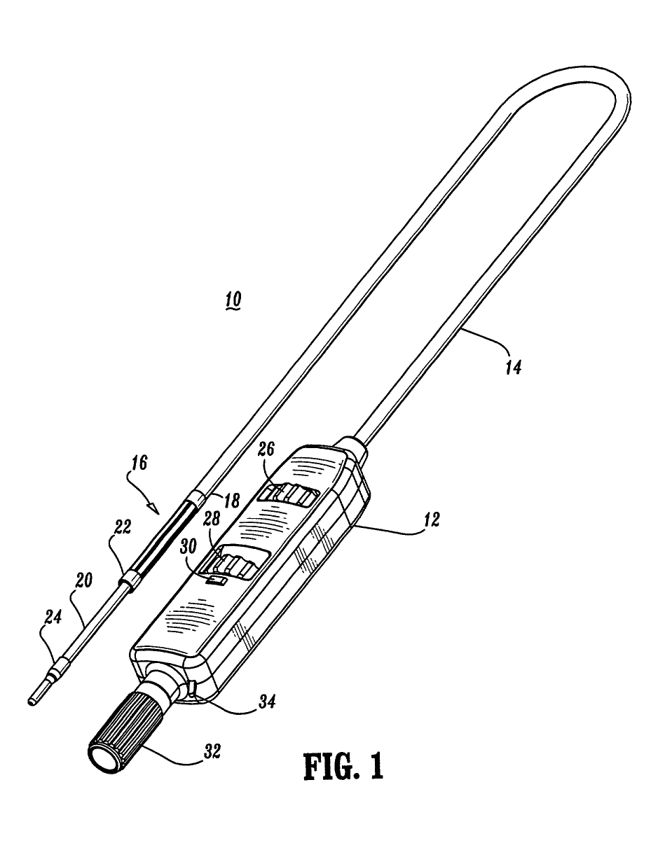

Referring to FIG. 1, there is illustrated a novel coil fastener applier 10 for use in installing graft material. In general, applier 10 generally includes a handle assembly 12 having an elongated flexible shaft 14 extending from a distal end 18 of the handle assembly 12. An onion assembly 16 is provided at the distal end 18 of elongated shaft 14. A coil fastener storage chamber 20 extends from a distal end 22 of the onion assembly 16 and terminates in an end cap 24.

Controls are provided on the handle assembly for various functions. A distal thumb wheel 26 is provided for expanding the onion assembly. A central thumb wheel 28 is provided for pivoting a fastener driving head 90° relative to the elongated shaft and rotating and indexing the fastener driving head between the various wings of the onion assembly. A lock tab 30 secures central thumb wheel 28 in position. An elongated proximal knob 32 is provided for extending a slide portion of the fastener driving head past the expanded onion assembly and for driving a fastener, such as, for example, a helical coil fastener out of the fastener driving head and through graft material and into tissue. A release lever 34 may also be provided on the handle assembly 12 for releasing the extended slide.

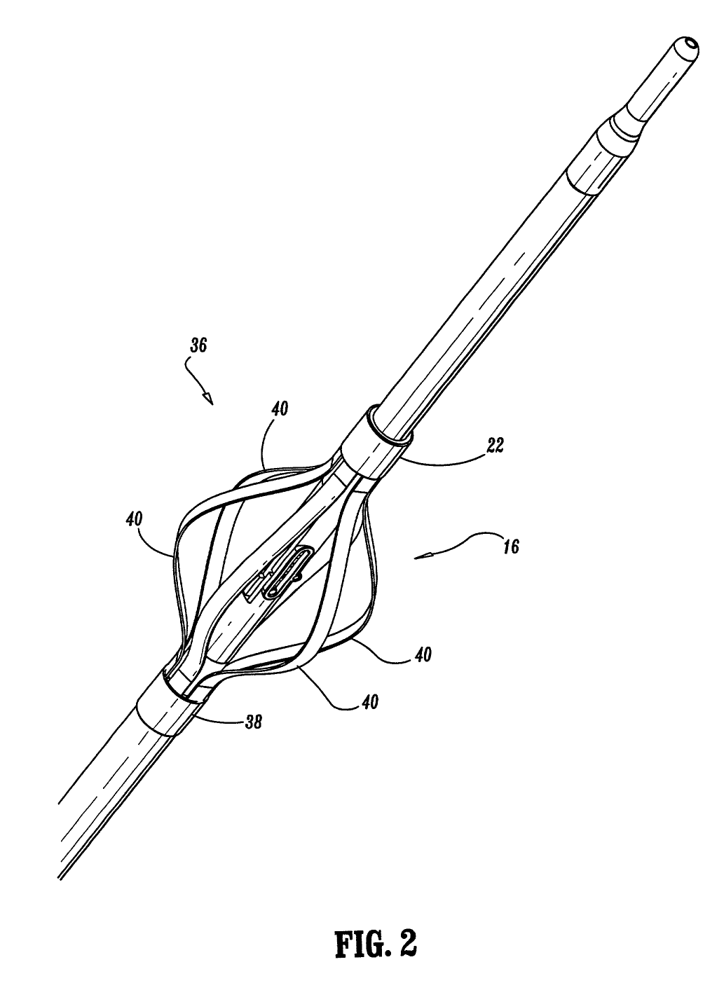

Referring to FIGS. 2-4, there is illustrated the distal end 36 of the applier 10 with the onion assembly 16 in an expanded condition. Onion assembly 16, having wings 40, is expanded in a conventional manner by moving an inner rod or wire connected to distal end 22 of onion assembly 16 relative to shaft 14 which is connected to a proximal end 38 of the onion assembly. A fastener driving head 42 is located within onion assembly 16.

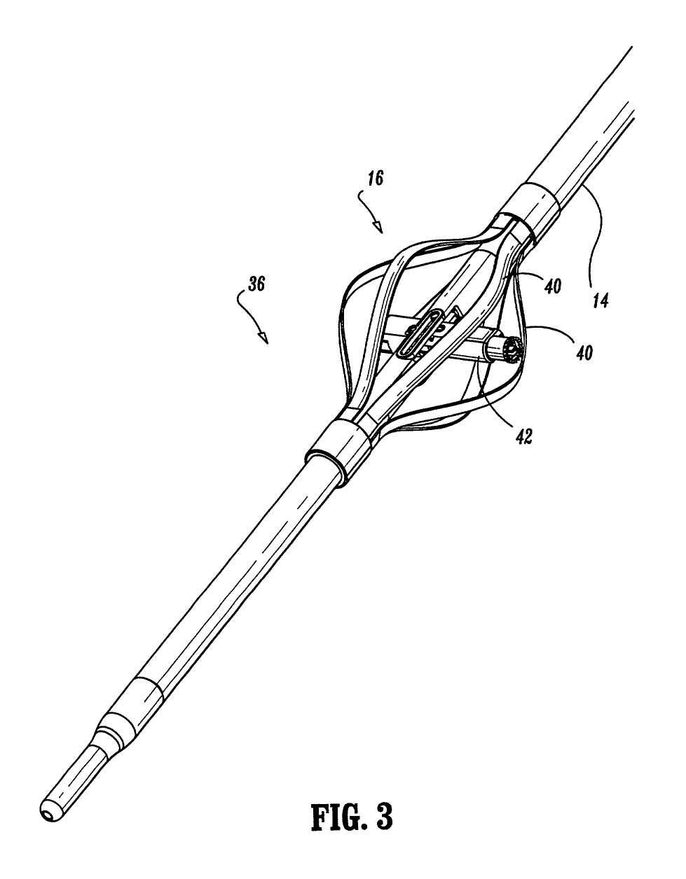

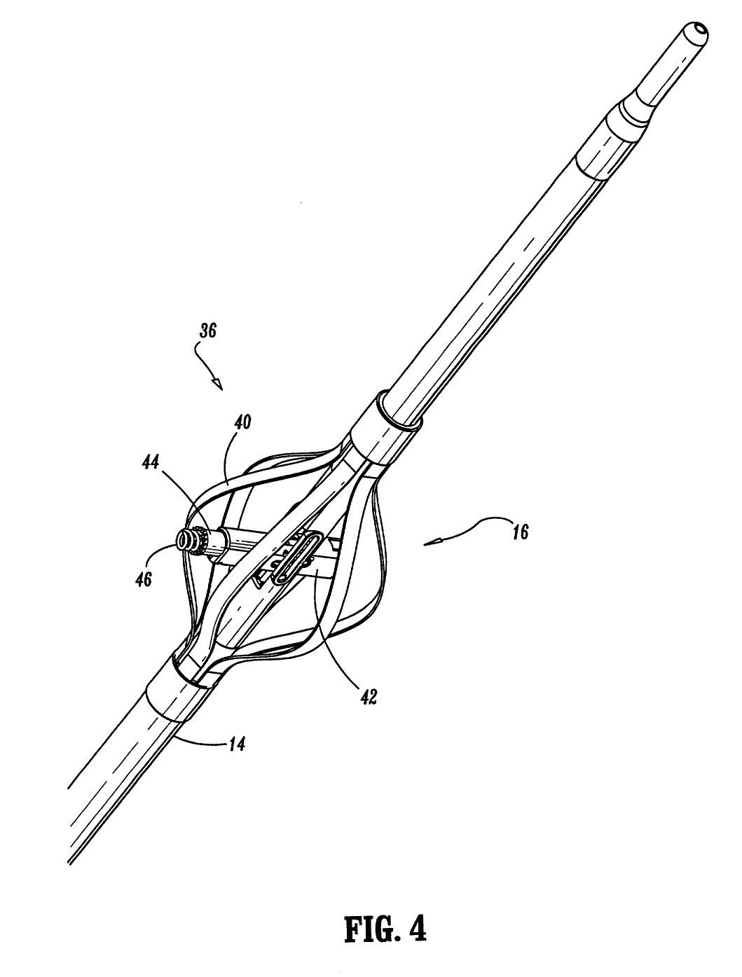

Referring to FIGS. 3-4, there is illustrated fastener driving head 42 pivoted 90° relative to elongated shaft 14.

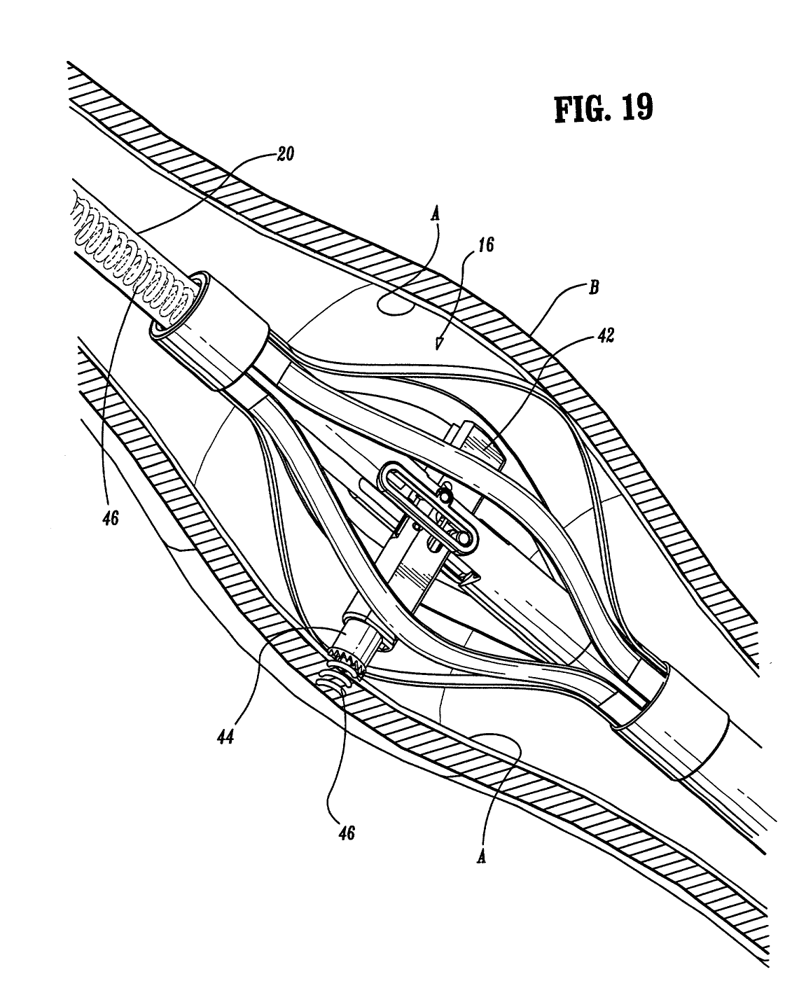

Specifically, referring to FIG. 4, there is illustrated fastener driving head 42 pivoted 90° relative to elongated shaft 14 and a slider 44 extended out of fastener driving head 42 to provide compression to graft material (FIG. 19). A helical coil fastener 46 is illustrated partially driven out of slider 44. This may be done to test the operation of applier 10 but, however, in use, the fastener 46 is not driven out of slider 44 until compression has been applied to the graft material.

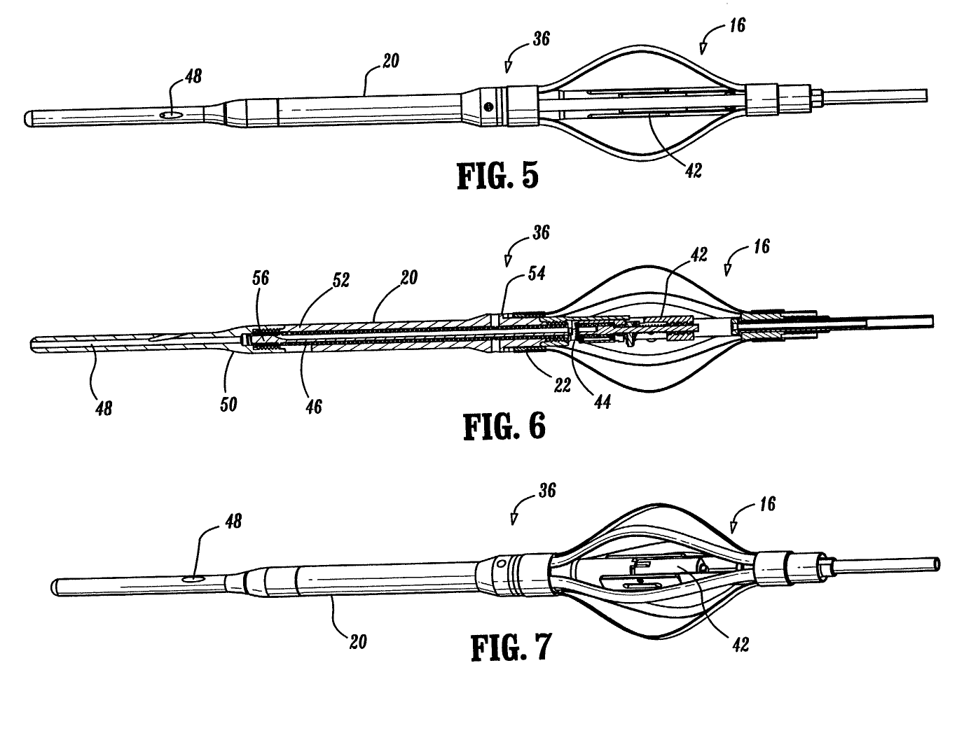

Referring to FIGS. 5-7, there is illustrated the distal end 36 of applicator 10 with the onion assembly 16 in an expanded condition. Referring to FIG. 6, there is illustrated the fastener driving head 42 in longitudinal alignment with the elongated shaft 10 and fastener storage chamber 20 which extends distally of the onion assembly 16. Chamber 20 is provided to contain a plurality of helical coil fasteners 46 for use with fastener driving head 42. With fastener driving head 42 in longitudinal alignment with chamber 20, slider 44 can be extended to remove and load a fastener 46 from chamber 20 into fastener head 42. Extending distally from the fastener storage chamber 20 is end cap 24. Preferably, end cap 24 includes a bore 48 therethrough for receipt of a conventional guidewire.

As shown, a proximal end 50 of end cap 24 is threaded to a distal end 52 of storage chamber 20 and a proximal end 54 of storage chamber 20 is threaded to distal end 22 of onion assembly 14. Thus, chamber 20 forms a detachable loading unit for use with applier 10. A plurality of helical coil fasteners are provided within chamber 20 about a mandrel 56.

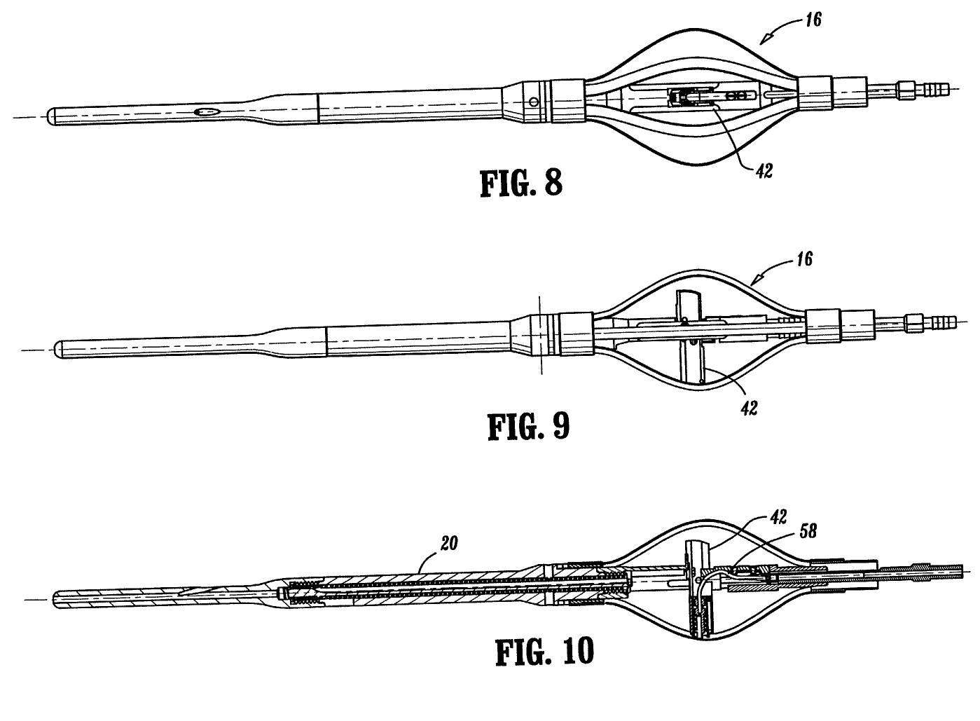

Referring to FIGS. 8-10, there is illustrated the distal end 36 of applier 10 with fastener driving head 42 pivoted 90° relative to elongated shaft 14. As specifically shown in FIG. 10, fastener driving head 42 is pivoted by means of a wire connected to control thumb wheel 28. A flexible, and preferably, shape memory wire 58 such as, for example, a Nitinol or Tinel wire which can be moved to extend and retract slider 44. Wire 58 can also be rotated within the elongated shaft and head 42 to drive a fastener 46 (positioned on a mandrel) into tissue. A proximal end of wire 58 is connected to proximal knob 32.

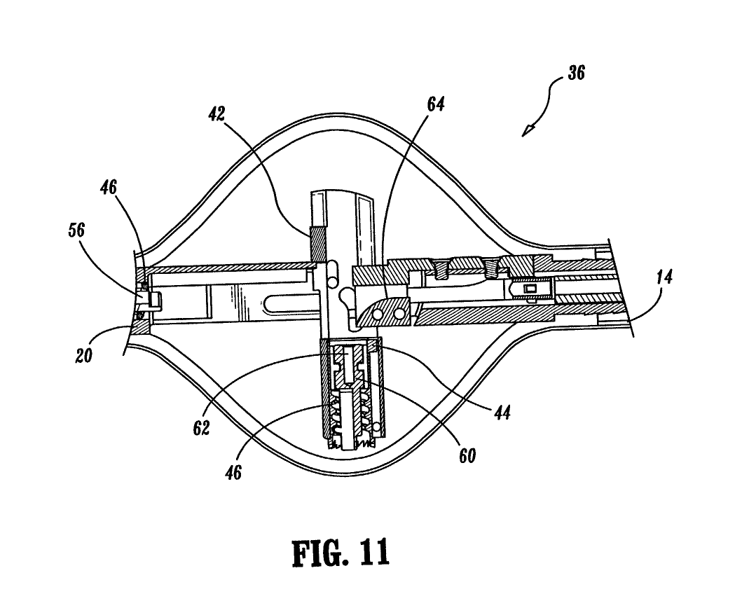

Referring to FIG. 11, there is illustrated the distal end 36 of applicator 10 with the fastener driving head 42 pivoted 90° relative to the elongated shaft 14. As illustrated, helical coil fastener 46 is contained within slider 44 and can be driven out of the slider 44 by a mandrel 60. Mandrel 60 includes a longitudinal bore 62 for receipt of an end of the driving wire (not shown). In addition to moving slider 44 by axially moving the wire within elongated shaft 14, helical coil fastener 46 can be rotated out of the fastener driving head 42 by rotation of the driving wire. A guide 64 is preferably provided within the distal end 18 of elongated shaft 14 so as to guide the driving wire around the 90° bend. It should be noted that to load a helical coil fastener 46 from the supply chamber 20 into the fastener driving head 42, the fastener driving head 42 is pivoted to be in longitudinal alignment with the elongated shaft 14 and thus slider 44 can then be extended such that the mandrel 60 contained therein contacts mandrel 56 in the storage chamber 20, and the wire rotated in a reverse direction to draw a helical coil fastener 46 out of the chamber 20 and into slider 44.

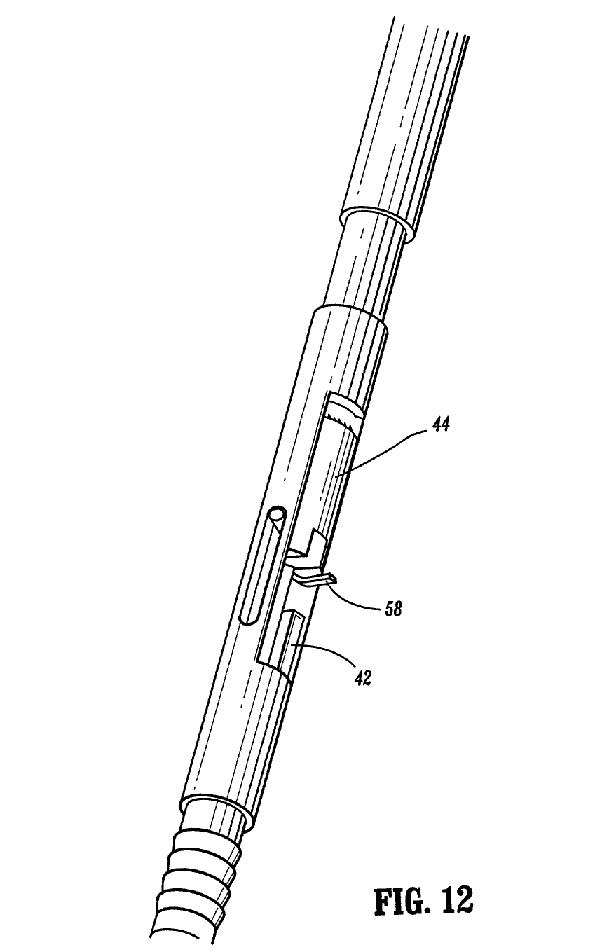

FIG. 12 illustrates the driving wire 58 within fastener driving head 42 and slider 44.

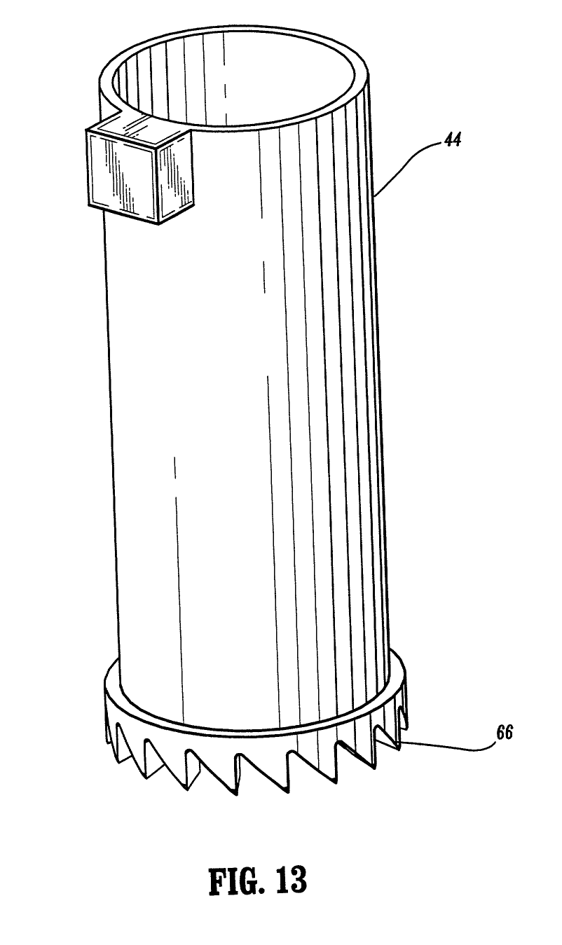

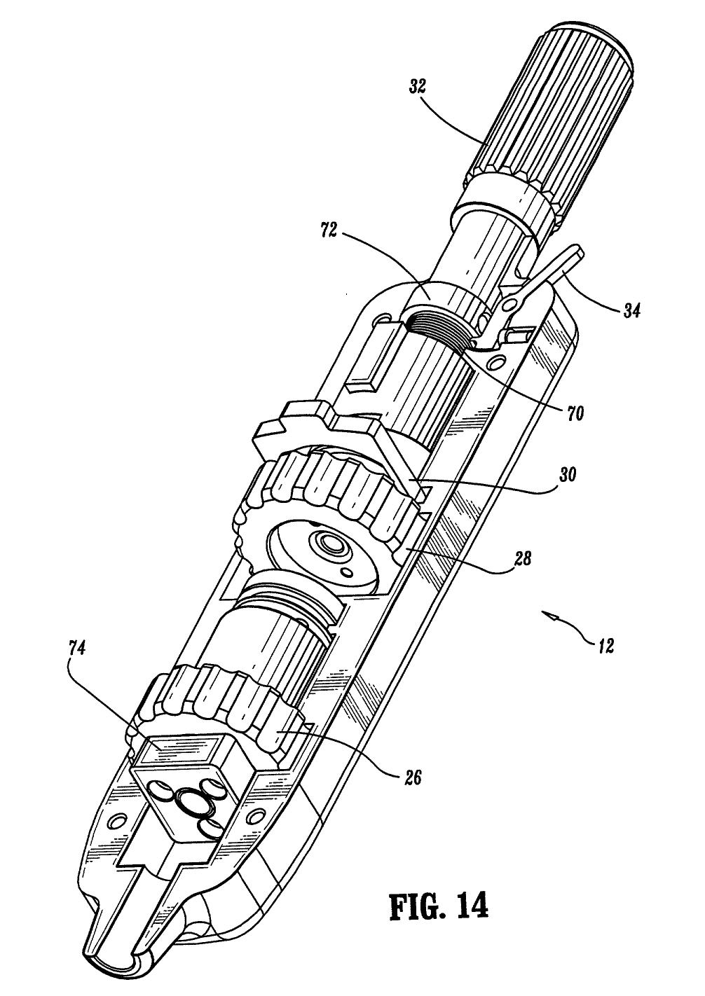

FIG. 13 illustrates an enlarged view of slider 44 which includes teeth 66 which are useful in engaging the graft material such that rotation of a coil fastener out of the slider does not rotate or disturb the graft material.

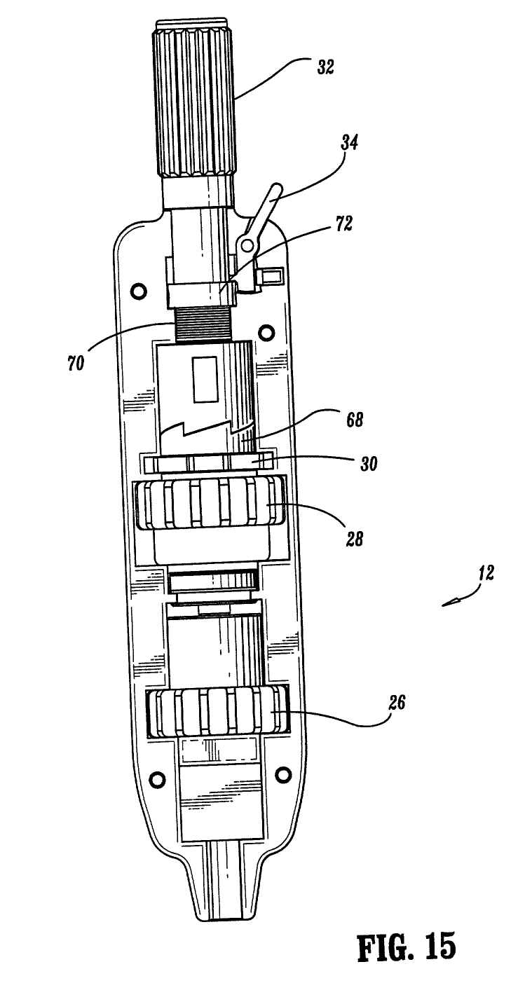

Referring now to FIGS. 14 and 15, there are illustrated views of the handle assembly 12 and associated controls. As noted above, controls are provided for operating the various functions. Distal thumb wheel 26 is provided for expanding the onion assembly. Central thumb wheel 28 is provided for pivoting fastener driving head 42 90° and indexing by 60° the fastener driving head 42 relative to the elongated shaft 14. The head 42 is pivoted 90° by moving central thumb wheel 28 longitudinally and indexed 60° by rotation of wheel 28. Indexing can only occur with head 42 in alignment with the shaft 14. Lock tab 30 adjacent the central wheel 28 locks and releases the fastener driving head 42 in the 90° position.

A ratchet feature 68 is provided with the central knob to control the indexing. Elongated proximal knob 32 is connected to a proximal end of wire 58 and is provided to move slider 44 in the fastener driving head 42 into engagement with graft material and/or tissue by longitudinal movement of knob 32. Rotation of the knob 32 rotates a helical coil fastener contained within the fastener driving head 42 out of the slider 44 and into tissue and is used to load a fastener 46 within the slider 44. A release lever 34 adjacent the proximal knob 32 is provided to maintain the slider in an extended position by releasably engaging an edge 72 of knob 32. Proximal knob 32 includes fine threads 70 for precisely rotating the driving wire 58. A guide block 74 is provided within assembly 12 to guide the various control wires or cables.

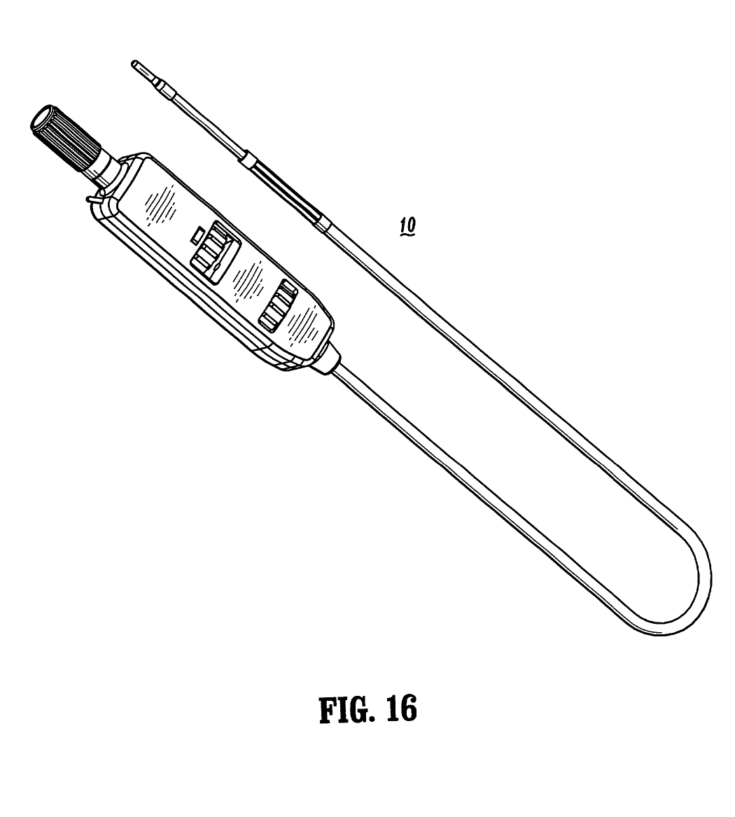

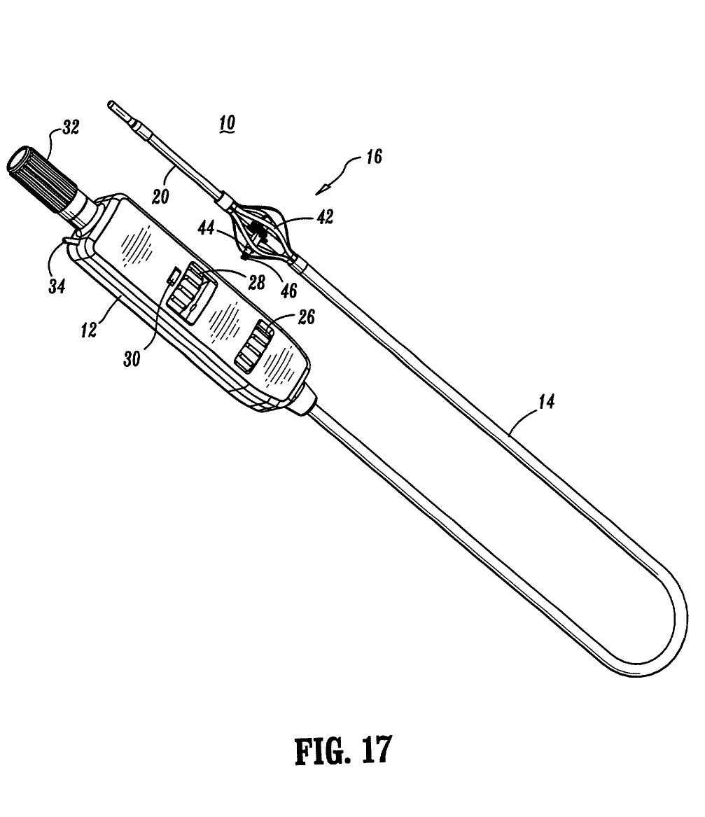

Referring now to FIGS. 16 and 17, prior to use, the control unit is actuated so as to draw a helical coil fastener out of the supply and into the slider. As shown in Sheet 19, operation of the onion assembly, pivoting heads and slider may be tested prior to use. The complete test, and in fact, operation procedure is as follows:

First rotate distal thumb wheel 26 to open and expand onion assembly 16. Next, rotate proximal knob 32 to load fastener 46 into mandrel 60. Then pull central wheel 28 and lock with lock tab 30 into position to pivot head 42 to 90° position. Next, push in (proximal) knob 32 by a desired amount and ratchet in place to extend slider 44. Rotate (proximal) knob 32 to fire fastener out of slider 44. Actuate release lever 34 to retract slider 44. Release lock tab 30 to pivot head 42 to return to in-line position. Rotate proximal knob 32 to reload pivoting head 42 with fastener 46. Rotate central wheel 28 in forward position to index pivoting head 42 and repeat firing sequence.

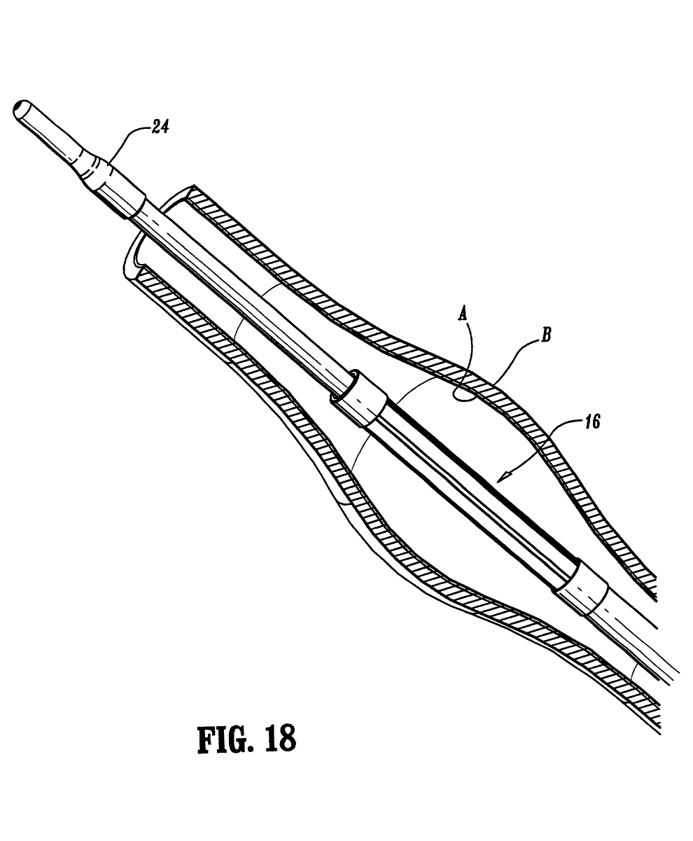

Referring to FIGS. 18 and 19, in use the distal end 36 of the applier 10, including onion assembly 16 is positioned at the aneurism site by advancing the end cap over a guidewire (not shown).

Referring to FIG. 19, once in position, the handle controls are actuated to expand the onion assembly 16 and pivot the fastener drive head 42 90° relative to the elongated shaft 14. Once pivoted 90°, slider 44 is extended out of the head 42 and into compressive engagement with graft material A positioned within the aneurism site. Subsequently, the handle assembly is actuated to rotate the helical coil fastener 46 out of slider 44 through the graft material A and into tissue B to secure the graft material A within the aneurism site.

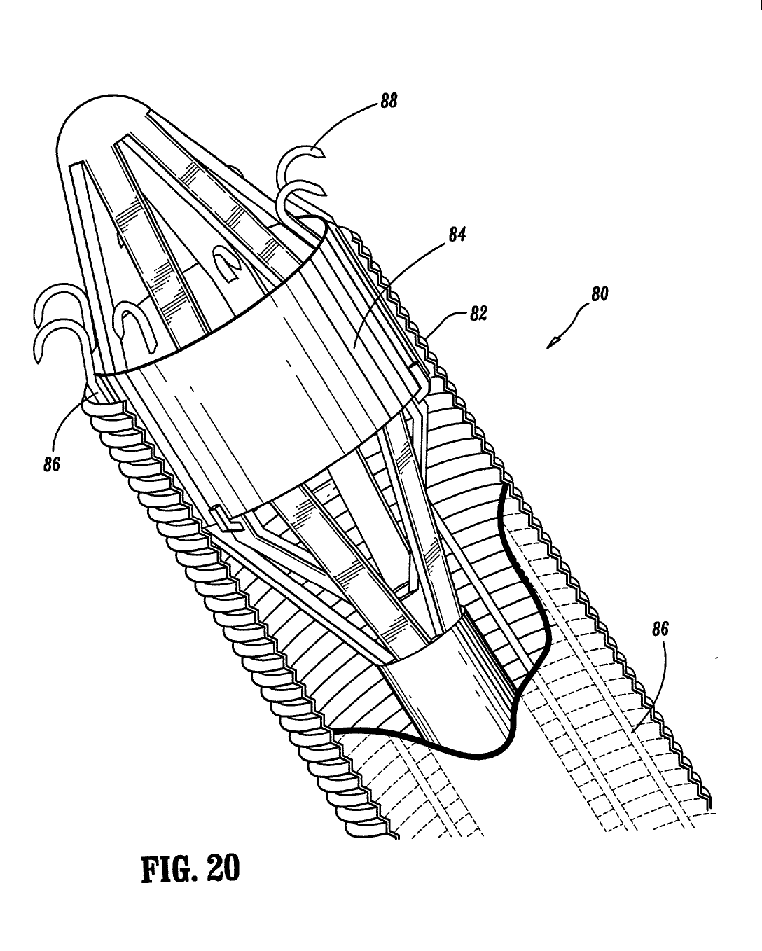

Referring now to FIG. 20, there is illustrated a novel graft assembly for use with the disclosed applicators. In particular, the graft assembly 80 includes a section of graft material 82 having a generally flat expandable Nitinol ring 84 positioned at one or both ends of the graft material 82. The Nitinol ring 84 is provided to maintain the graft material 82 in expanded engagement with the walls of the tissue section or a vessel during the fastening operation. Nitinol ring 84 may have various other configurations such as round, narrow, etc.

The Nitinol ring 84 may be supported about the onion assembly by additional arms, associated with the onion assembly, which flex to an open condition as the onion assembly is expanded within the graft material. Additionally, there may also be provided longitudinal struts 86 affixed within graft material 82 and terminating in hooks 88. Hooks 88 are configured such that upon expansion of the onion assembly and thus the Nitinol ring 84, hooks 88 are driven into tissue to assist in preventing longitudinal migration of the graft assembly 80 within the aneurism site alone or in addition to affixing the graft assembly 80 with helical coil fasteners or other staple type fasteners. Graft material 82 may be of a suitable known material, such as, for example, Bifurcated Arterial Graft Arterial Graft Surgical Mesh. Struts 86 and hooks 88 may be formed of various suitable biocompatible material such as polymers, and/or stainless steel, or other similar metals.

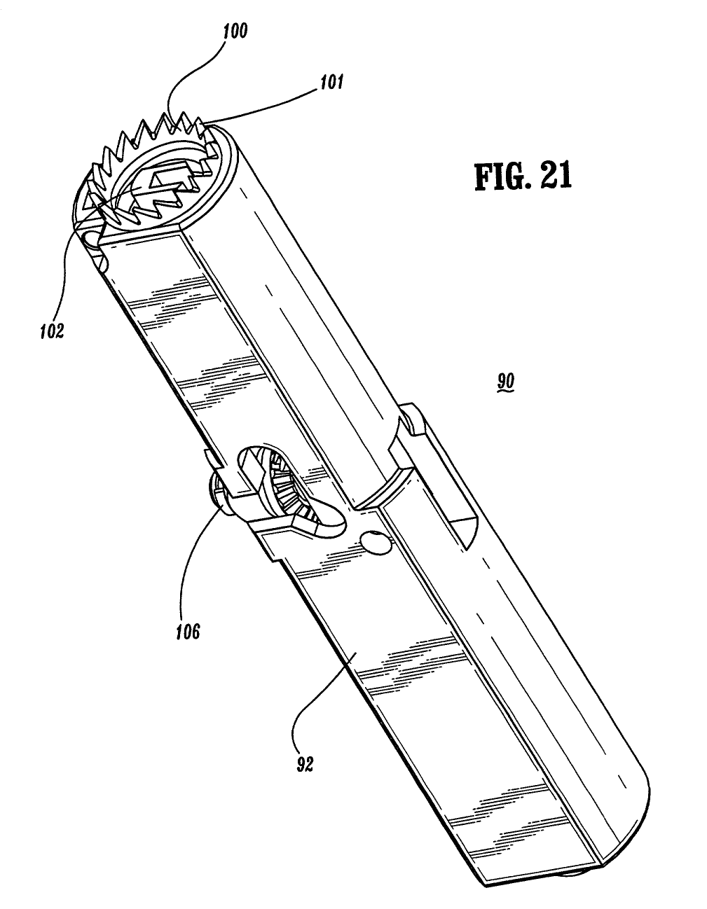

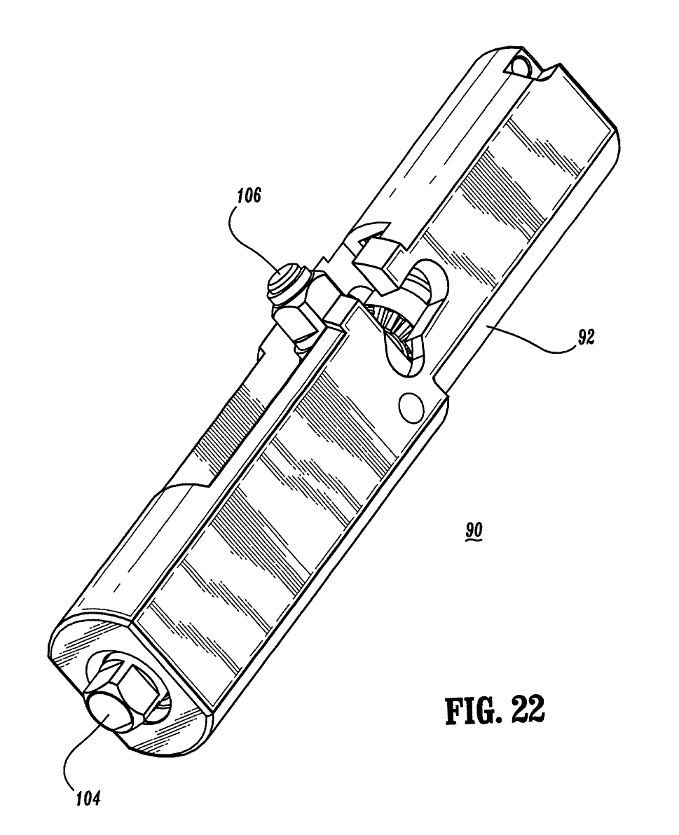

Referring to FIGS. 21-24, there is illustrated an alternative embodiment of a pivoting and driving head assembly for use with the disclosed fastener applier 90. Head assembly 90 includes a housing 92 which is pivotally mounted on shaft 124. Instead of utilizing a Nitinol wire to pivot a staple driving head 90° relative to the elongated shaft 14, a pair of beveled gears 94 and 96 and a clutch mechanism 98 are provided to pivot the head and drive a helical coil fastener out of slider 100. Clutch mechanism 98 may be provided to engage and disengage the driving mechanism with fastener driving head 90.

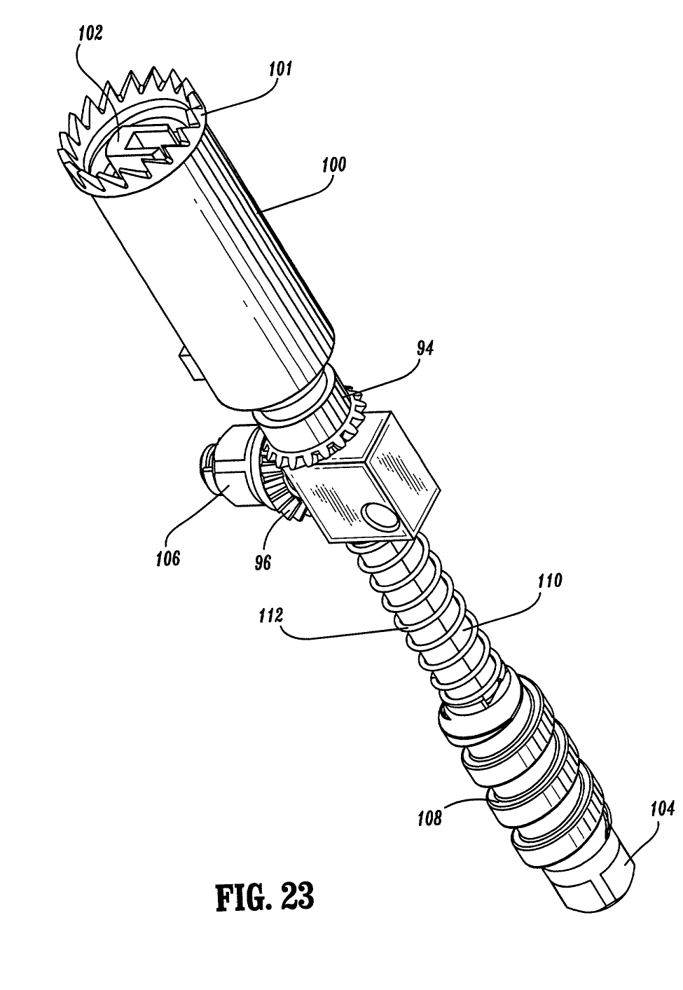

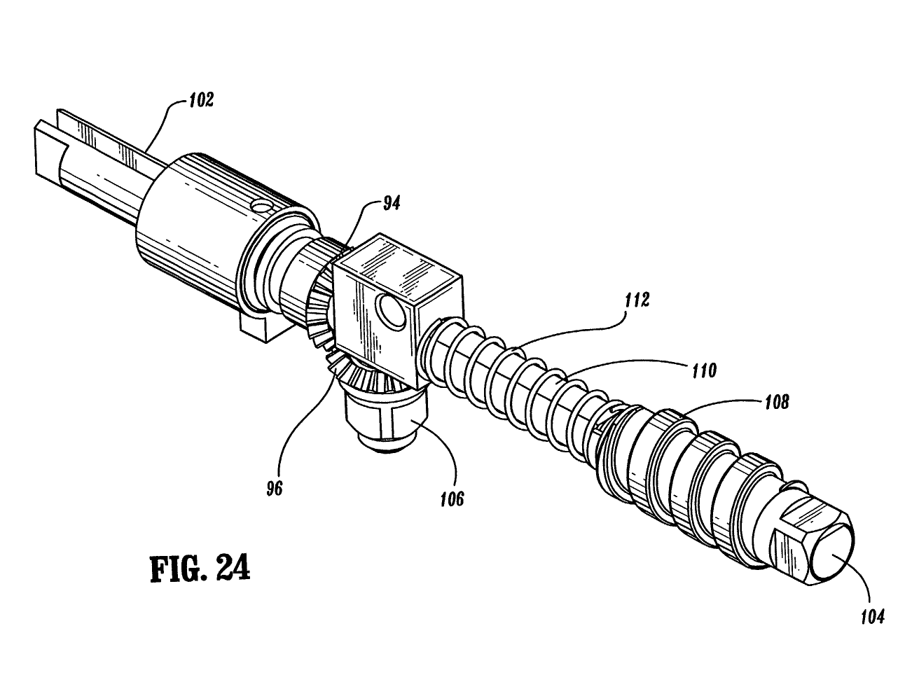

With regard to FIGS. 21-24, head assembly 90 generally includes a housing 92 enclosing a first beveled gear 94 and a second beveled gear 96. A clutch mechanism 98 is provided within housing 92. As shown in FIG. 21, head assembly 90 also includes a slider 100 having slider teeth 101 and a staple driving mandrel 102. Referring to FIGS. 23 and 24, wherein housing 92 has been removed, head assembly 90 includes a loading connecting nut 104 and a firing connecting nut 106. Connecting nuts 104 and 106 are configured to releasably engage a drive mechanism associated with a fastener applier such that when head assembly 90 is in longitudinal alignment with elongated shaft 14, drive mechanism will engage loading connecting nut 104 to facilitate loading a fastener into head assembly 90 and when head assembly 90 is pivoted 90° the actuating mechanism will engage firing connecting nut 106 to rotate the mandrel 02 and advance the fastener out of head assembly 90. As the fastener is being driven out of head assembly 90 into the graft, the shaft assembly moves forward. At the end of the stroke, the threaded sleeve of the clutch mechanism used to thread in pivot head housing and rotates until changing the direction of rotation. As shown, threaded sleeve 108 surrounds a generally D-shaped shaft 110 and is biased by a spring 112.

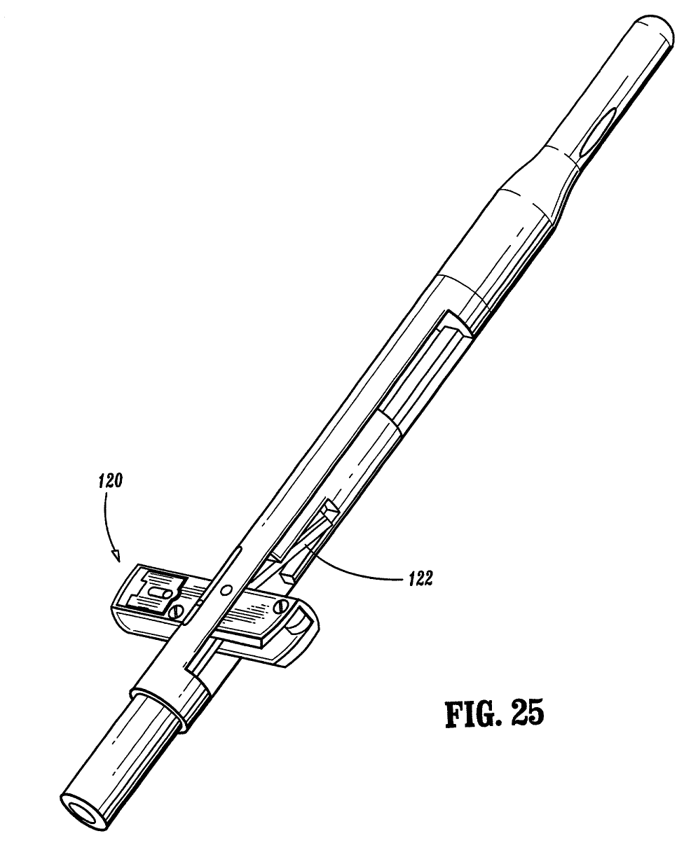

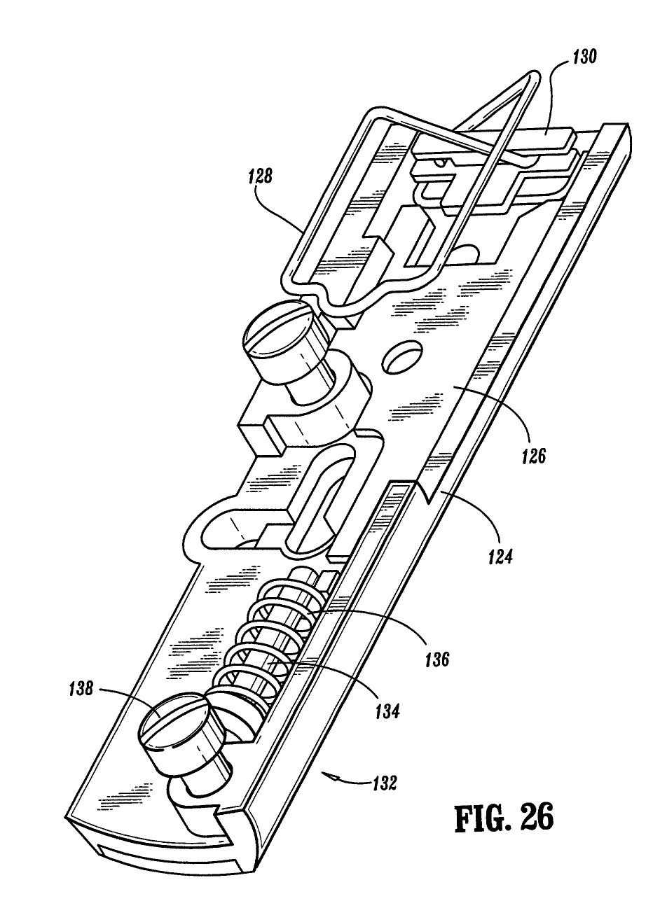

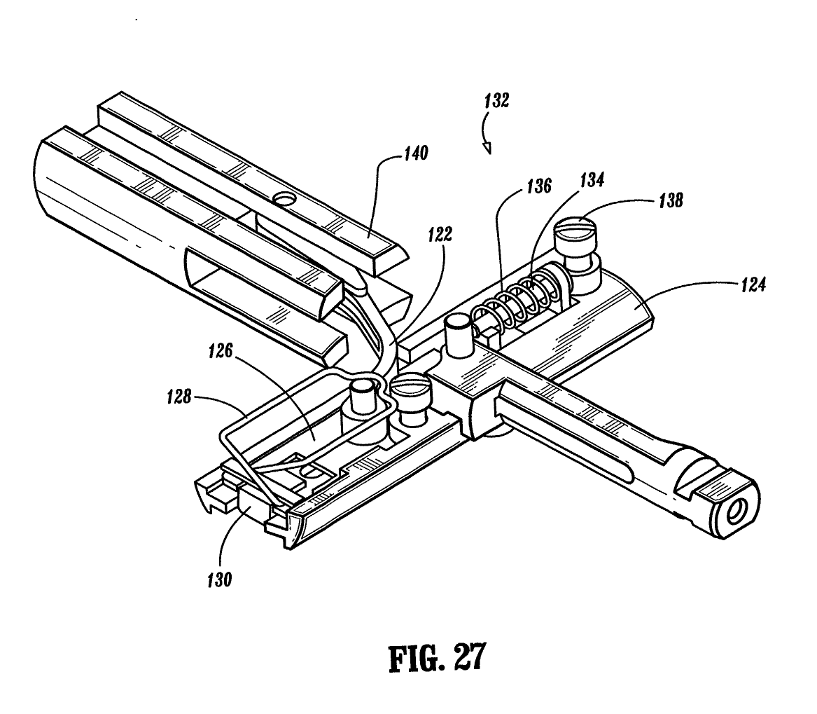

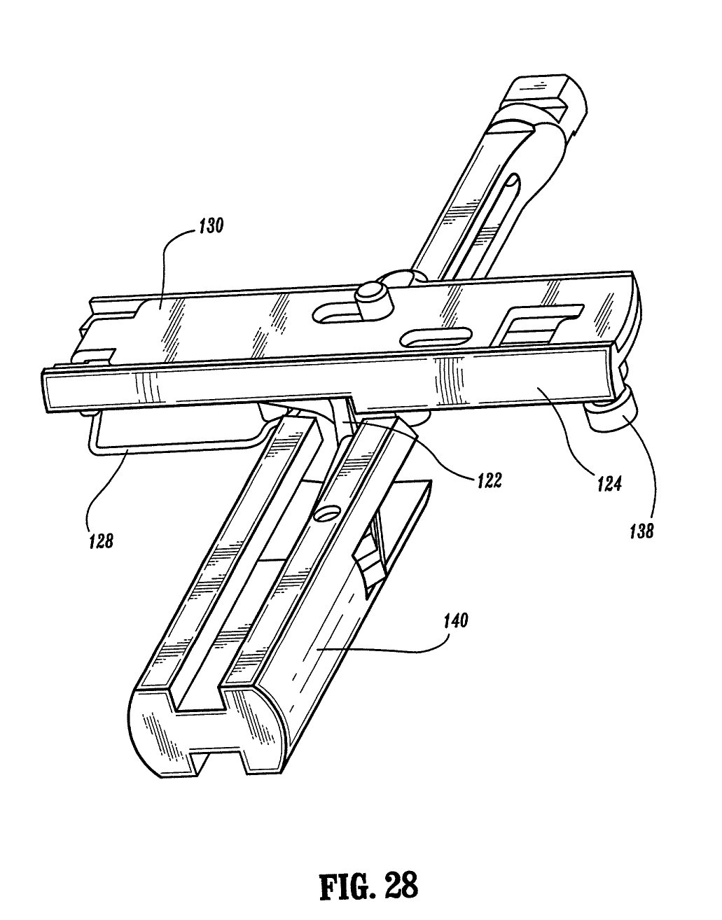

Referring now to FIGS. 25-28, there are illustrated various views of an alternative staple driving head 120. In particular, this embodiment of a staple driving head utilizes conventional staples in conjunction with a staple driver and an anvil to secure graft material within a vessel. The head 120 includes links 122 to pivot the head 90° relative to an associated shaft. As specifically shown in FIG. 26, the staple driving head 120 includes a body portion 124 having a longitudinally movable staple driving or pusher plate 126, and a feed spring 128 for biasing a staple stack toward an anvil 130. A tension device 132 having a rod 134, a spring 136 around rod 134, and an adjustment screw 138 are provided to return head 120 from a pivoted 90° position to a position longitudinally aligned with a distal end of a modified shaft 140 (FIG. 27). While not specifically illustrated, it is contemplated that a stack or plurality of conventional staples may be provided adjacent the staple biasing plate 126 and anvil 130 to provide a supply of more than one staples within the staple driving head.

Each of the alternative staple driving heads illustrated in FIGS. 21-24 and FIGS. 25-28 may be utilized in a manner similar to that discussed with respect to FIGS. 1-19 for securing a graft material within an aneurism site.

What is claimed is:

1. An endovascular fastener applicator for endoluminally fastening a prosthetic graft to a vessel with at least one fastener comprising:
a tubular body configured for positioning within a vessel;
an expandable portion disposed adjacent a distal end of the tubular body and being expandable to support a prosthetic in contact with an inner surface of a vessel;
a fastener applying head rotatably mounted on the distal end of the tubular body and movable between a load position longitudinally aligned with the tubular and a firing position oriented approximately 90° with respect to the tubular body; and
a handle assembly mounted on a proximal end of the tubular body.

2. The applicator as recited in claim 1, wherein the handle assembly has a first control to expand the expandable portion.

3. The applicator as recited in claim 1, wherein the handle assembly includes a second control to pivot the fastener driving head to the firing position.

4. The applicator as recited in claim 3, wherein the second control also rotates the fastener driving head about the longitudinal axis of the tubular body.

5. The applicator as recited in claim 3, wherein the handle assembly includes a third control to move a fastener out of the fastener driving head and into tissue.

6. The applicator as recited in claim 5, wherein the third control further moves a fastener carrying slider into engagement with tissue.

7. The applicator as recited in claim 1, wherein the fastener is a helical coil fastener.

8. The applicator has recited in claim 7, further comprising a storage chamber extending from a distal end of the expandable portion, the storage chamber containing at least one helical coil fastener.

9. The applicator as recited in claim 3, wherein the third control is connected to the fastener carrying slider by a wire formed of a shape memory material.

10. The applicator as recited in claim 1, wherein the fastener is a conventional staple.
